(19) United States
(12) Patent Application Publication
(10) Pub. No.: US 2009/0098187 A1
(43) Pub. Date: Apr. 16, 2009

(54) Composition And Its Use For The Manufacture Of A Medicament For Treating, Prophylactically Treating, Preventing Cancer And/Or Infections In The Urinary Tract

(76) Inventors: Tor Peters, Schaffhausen (CH); Peter Wiklund, Lidingo (SE)

Correspondence Address: INSKEEP INTELLECTUAL PROPERTY GROUP, INC, 2281 W. 190TH STREET, SUITE 200, TORRANCE, CA, 90504, US

(21) Appl. No.: 12/093,572

(22) PCT Filed: Nov. 15, 2006
(86) PCT No.: PCT/EP2006/010924

§ 371 Date: Oct. 8, 2008

Related U.S. Application Data
(60) Provisional application No. 60/736,826, filed on Nov. 15, 2005.

(30) Foreign Application Priority Data
Nov. 14, 2005 (EP) 05024815.2

(51) Int. Cl. A61L 27/54; C01B 21/24; A61K 33/00; C08K 3/28; A61P 35/00

(52) U.S. Cl. 424/425; 423/405; 424/486; 424/718; 524/429

(57) ABSTRACT

A composition is provided that allows for treatment of cancer, prophylactic treatment, and treatment of infection in the urinary tract. The composition comprises a nitric oxide (NO) eluting polymer that elutes nitric oxide (NO) in a therapeutic dose. The nitric oxide (NO) eluting polymer may be integrated with a carrier material, such that said carrier material, in use, regulates and controls the elution of said therapeutic dosage of nitric oxide (NO). The nitric oxide (NO) eluting polymer may be provided as a Solution or Suspension. Furthermore, a manufacturing method for said composition is disclosed, as well as uses of said composition in the urinary tract.

The present invention claims benefit of International Application No. PCT/EP2006/010924, filed 14 Nov. 2006 entitled Composition And Its Use For The Manufacture Of A Medicament For Treating, Prophylactically Treating, Preventing Cancer And/Or Infections In The Urinary Tract; which claims priority from European Patent Application No. 05024815.2 filed 14 Nov. 2005 entitled Composition, And Use Thereof, In Respect Of Cancer, Prophylactic Treatment And Infection In The Urinary Tract, Involving Nitric Oxide and U.S. Provisional Patent Application No. 60/736,826 filed 15 Nov. 2005 entitled Composition, And Use Thereof, In Respect Of Cancer, Prophylactic Treatment And Infection In The Urinary Tract, Involving Nitric Oxide, all of which are incorporated herein by reference.

### FIELD OF THE INVENTION

### Background of the Invention

This invention pertains in general to the field of treatment of cancer in the urinary tract, prophylactic treatment of the urinary tract, or therapy of infection in the urinary tract. More particularly the invention relates to a composition for treatment and prevention of cancer in the urinary tract, and a process for manufacturing of said composition, involving the use of nitric oxide (NO). Also, the present invention pertains to the use of NO for treatment and/or prevention of cancer in the urinary tract.

Cancer in the urinary bladder is the fourth most common malignancy among men, and the eighth most frequent among women. An average of approximately 300,000 new cases of cancers in the urinary bladder are diagnosed world wide every year. Of these are 90% of Transitional Cell Carcinoma (TCC) type, originating in the epithelial cells (the internal lining) of the bladder wall. When the tumor is limited to this layer, it is called superficial cancer in the urinary bladder. This type of cancer tends to recur despite surgery and treatments.

Today, the most common way of treating cancer in the urinary bladder is to surgically remove the tumor(s) under anesthesia (partial or general). Most of the time such surgery is performed through the urethra of the patient. If numerous tumors are present the physician often is compelled to perform many such surgeries or a more extensive operation, in which a partial or complete removal of the urinary bladder is required, especially if the cancer has migrated to the muscle layer of the urinary bladder. Surgery, no matter in what extent, is always a very trying and cumbersome experience for the body and patient on which the surgery is performed. It always results in some time of recovery and period of convalescence. After having ascertained the type of tumor present in the urinary bladder through evaluation of the performed surgery, the urinary bladder may be flushed with chemotherapeutic materials to thereby destroy cancer cells that were not removed during surgery. This is done to kill off remaining cancer cells and to prevent growth of such remaining cancer cells. The use of chemotherapeutic materials involves the issue that chemotherapeutic materials not only destroy cancer cells but also healthy cells, which leads to deteriorated immune defense of the patient being treated.

It is known that nitric oxide (NO) provides an alternative to conventional therapies, such as antibiotics. Nitric oxide is a highly reactive molecule that is involved in many cell functions. In fact, nitric oxide plays a crucial role in the immune system and is utilized as an effector molecule by macrophages to protect itself against a number of pathogens, such as fungi, viruses, bacteria etc.

NO is also known to have an anti-pathogenic effect, and furthermore NO has an anti-cancerous effect, as it is cytotoxic and cytostatic in therapeutic concentrations, i.e. it has among other effects tumoricidal and bactericidal effects. NO has for instance cytotoxic effects on human hematological malignant cells from patients with leukemia or lymphoma, whereby NO may be used as a chemotherapeutic agent for treating such hematological disorders, even when the cells have become resistant to conventional anti-cancer drugs. This anti-pathogenic and anti-tumor effect of NO is taken advantage of by the present invention, resulting in fewer and milder adverse effects as for instance many anti-cancer drugs.

However, due to the short half-life of NO, it has hitherto been very hard to treat cancers for a sufficient period of time to obtain results. This is because NO is actually toxic in high concentrations and has negative effects when applied in too large amounts to the body. NO is also a vasodilator, and too large amounts of NO introduced into the body will cause a marked reduction of the blood pressure that may result in a complete collapse of the circulatory system. On the other hand, NO has a very short half-life of fractions of a second up to a few seconds, once it is released. Hence, administration limitations due to short half-life and toxicity of NO have been limiting factors in the use of NO in the field of anti-pathogenic and anti-cancerous treatment so far.

The urinary tract including urine is a unique environment in respect of treatment with nitric oxide, since the end oxidation product of nitric oxide is NO2-. In almost all other biological tissues nitric oxide is oxidized to NO3-, since there is hemoproteins, such as hemoglobin, present. This effect results in that very high concentrations of nitric oxide may be obtained during long periods of time in the urinary bladder, since the nitric oxide that is eluted in urinary bladder is first oxidized to N02-, which in return is reduced back to nitric oxide. This effect may be observed already at a pH of 7.5, but is strongly amplified at lower pH.

In recent years research has been directed to polymers with the capability of releasing nitrogen oxide when getting in contact with water. Such polymers are for example polyalkyleneimines, such as L-PEI (Linear PolyEthylenelmine) and B-PEI (Branched PolyEthylenelmine), which polymers have the advantage of being biocompatible before and after the release of nitrogen oxide.

Other examples for NO eluting polymers are given in U.S. Pat. No. 5,770,645, wherein polymers derivatized with at least one —NOX group per 1200 atomic mass unit of the polymer are disclosed, X being one or two. One example is an S-nitro-sylated polymer and is prepared by reacting a polythiolated polymer with a nitrosylating agent under conditions suitable for nitrosylating free thiol groups.

Akron University has developed an NO-eluting L-PEI molecule that can be spun onto the surface of medical devices to be permanently implanted in the body, such as implanted grafts, showing significant improvement of the healing process and reduced inflammation when implanting such devices. For instance, U.S. Pat. No. 6,737,447 of Akron University discloses a coating for medical devices that provides nitric oxide delivery using nanofibers of linear poly(ethylenimine)-diazeniumdiolate. Linear poly(ethylenimine)-diazeniumdiolate releases nitric oxide (NO) in a controlled manner to tissues and organs to aid the healing process and to prevent injury to tissues at risk of injury. Electrospun nano-fibers of linear poly(ethylenimine)-diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of the device. However, the disclosure of U.S. Pat. No. 6,737,447 is silent concerning an improvement of present technology in respect of therapeutic treatments in the urinary bladder with nitric oxide. Moreover, the meaning of controlled in the context of U.S. Pat. No. 6,737,447 is only directed to the fact that nitric oxide is eluted from the coating during a period of time. Therefore, the interpretation of controlled in respect of U.S. Pat. No. 6,737,447 is different from the meaning of regulating in the present context. Regulate, according to the present context is intended to be interpreted as the possibility to vary the elution of nitric oxide to thereby achieve different elution profiles.

US 2003/0050256 discloses a sugar modified SIN-1 composition for generating nitric oxide in response to hydrolytic activity of a glycosidase. This composition is suited for killing cancerous cells in for example the bladder. However, US 2003/0050256 fails to disclose a nitric oxide eluting polymer. Furthermore, the sugar modified SIN-1 is in need of a glycosidase to deliberate nitric oxide. The disclosure according to US 2003/0050256 takes advantage of the role of carbohydrates in cell recognition and internalization processes. Thus, the sugar modified SIN-1 is just sugar modified. The sugar moiety ensures that the nitric oxide donor compound, i.e. the sugar modified SIN-1, is inactive until it encounters an appropriate enzyme, such as glycosidase. Moreover, the sugar moiety may be designed to suit a specific receptor or surface ligand. However, it is less advantageous that the composition is in need of a specific enzyme to start the elution of nitric oxide. Hence, there is a need for improved delivery of nitric oxide to the environment of the bladder.

U.S. Pat. No. 5,770,645 discloses the delivery of nitric oxide to prevent vasospasms post hemorrhage and to treat bladder irritability, urethral structures and biliary spasms, nitric oxide is released from S-nitrosylated polymers in hydrogels, via injection.

WO 95/09612 and U.S. Pat. No. 5,814,666 disclose compositions capable of releasing nitric oxide. The compositions comprise one or more nitric oxide generators, preferably encapsulated in vesicles, such as liposomes. These compounds are capable of releasing nitric oxide in an aqueous solution. Polyethylenimine is mentioned as suitable for use in this respect. WO 95/09612 and U.S. Pat. No. 5,814,666 fail to disclose the use of such compounds for the use as a medicament in the treatment of cancer in the urinary tract or for the use as a medicament in the treatment of cancer in the epithelial cells in the urinary tract. Furthermore, WO 95/09612 and U.S. Pat. No. 5,814,666 fail to disclose a beneficial effect in the bladder, since the disclosed compounds preferably are encapsulated in liposomes. The encapsulation in liposomes is done to ensure that the elution of nitric oxide is not initiated before the liposomes are disrupted, i.e. when ingested in macrophages. This is also why it is preferred that the compounds disclosed in WO 95/09612 and U.S. Pat. No. 5,814,666 are injected into a cancerous tumor. In the urinary tract it is less advantageous that nitric oxide may only be eluted after the liposomes have been degenerated. More precisely, elution of nitric oxide is only made possible in the urinary tract if also a suitable enzyme is provided.

Hence, there is a need for a nitric oxide eluting composition facilitating delivery of nitric oxide in the urinary tract, providing regulated, e.g. delayed, spontaneous, instantaneous or immediate, elution of nitric oxide at that location.

An improved composition for the treatment and/or prevention of cancer in the urinary tract, prophylactic treatment of the urinary tract, and therapy of infection of the urinary tract, which composition presents the possibility to treat and/or prevent such disorders, does not involve a surgical step, does not involve the use of chemotherapeutic materials, does not cause resistance against the active pharmaceutical substance, is easy to apply, provides improved circulation in form of a vasodilating effect, provides a painless treatment, has fast inset of treatment effect, would be advantageous.

### OBJECTS AND SUMMARY OF THE INVENTION

Accordingly, the present invention seeks to mitigate, alleviate or eliminate according to certain embodiments amongst others one or more of the above-identified deficiencies in the art and disadvantages singly or in any combination and deals with the disadvantageous issues of the art mentioned above, by providing a composition, manufacturing method or process for the composition and uses of the composition according to the appended patent claims.

Various aspects of the invention are recited in the attached independent claims.

According to one aspect of the invention, a composition is provided that allows for treatment of cancer in the urinary tract, prophylactic treatment of the urinary tract, or treatment of infection in the urinary tract. The composition comprises a nitric oxide (NO) eluting polymer arranged to contact the area to be treated, such that a therapeutic dose of nitric oxide is eluted from said nitric oxide eluting polymer to said area.

According to another aspect of the invention, a manufacturing process for such a composition is provided, wherein the process is a process for forming a composition that allows for target treatment of cancer in the urinary tract, prophylactic treatment of the urinary tract, and treatment of infection in the urinary tract. The process comprises selecting a nitric oxide eluting polymer, such as nano fibers, fibers, nano-particles, micro-spheres, or powder, and deploying said nitric oxide eluting particles into forms such as nano-particles, micro-spheres, or powder to be comprised in said composition.

According to still another aspect of the present invention use of nitric oxide for manufacturing of a medicament to treat and/or prevent cancer in the urinary tract is provided.

Further embodiments are subject of the dependent claims.

The present invention provides for the possibility to treat and/or prevent cancer in the urinary tract, prophylactic treatment of the urinary tract, or infection of the urinary tract, which does not involve a surgical step, does not involve the use of chemotherapeutic materials, does not cause resistance against the active pharmaceutical substance, and still is easy to apply, provides improved circulation in form of a vasodilating effect, provides a painless treatment, and has fast inset of treatment effect, by the exposure of a urinary tract to NO, whereby a very effective anti-cancer therapy, prophylactic treatment, and/or anti-infection therapy of the urinary tract is achievable.

Hence, an advantageous delivery of nitric oxide from a nitric oxide eluting polymer in the urinary tract is provided, as regulated, e.g. delayed, spontaneous, instantaneous or immediate, elution of nitric oxide in the urinary tract is provided. This is accomplished since the urinary tract provides an environment in which the elution of nitric oxide from the polymer incorporated in embodiments of the present invention is very profitable, since the presence of high yields of nitric oxide may be maintained during long periods of time.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The following description focuses on embodiments, which for instance are applicable to a composition, in form of nano-particles, micro-spheres, or a powder of a polymer material, which allows for target treatment of cancer in the urinary tract, prophylactic treatment of the urinary tract, and treatment of infection in the urinary tract, amongst others.

The term urinary tract is in the present context intended to be interpreted as including the urinary bladder, the ureter, the urethra, and the renal pelvis.

With regard to nitric oxide (nitrogen monoxide, NO), its physiological and pharmacological roles have attracted much attention and thus have been studied. NO is synthesized from L-arginine as the substrate by nitric oxide synthase (NOS). NOS is classified into a constitutive enzyme, cNOS, which is present even in the normal state of a living body and an inducible enzyme, iNOS, which is produced in a large amount in response to a certain stimulus. It is known that, as compared with the concentration of NO produced by cNOS, the concentration of NO produced by iNOS is several orders higher, and that iNOS produces an extremely large amount of NO.

In the case of the generation of a large amount of NO as in the case of the production by iNOS, it is known that NO may react with active oxygen to attack exogenous microorganisms, such as bacteria and parasites, and cancer cells, but also to cause inflammation and tissue injury. On the other hand, in the case of the generation of a small amount of NO as in the case of the production by cNOS, it is considered that NO takes charge of various protective actions for a living body through the guanylate cyclase/GMP pathway (cGMP), such as vasodilator action, improvement of the blood circulation, antiplatelet-aggregating action, acceleration of the absorption in the digestive tract, regulation of renal function, neurotransmitter action, erection (reproduction), learning, appetite, and the like. Heretofore, inhibitors of the enzymatic activity of NOS have been examined for the purpose of preventing inflammation and tissue injury, which are considered to be attributable to NO generated in a large amount in a living body. However, the promotion of the enzymatic activity (or expressed amount) of NOS (in particular, cNOS) has not been examined for the purpose of exhibiting various protective actions for a living body by promoting the enzymatic activity of NOS and producing NO appropriately.

In recent years research has been directed to polymers with the capability of releasing nitrogen oxide when getting in contact with water. Such polymers are for example polyalkyleneimines, such as L-PEI (Linear PolyEthylenelmine), B-PEI (Branched PolyEthylenelmine), and PEI-C (PolyEthylenelmine Cellulose, which is a complex of polyethyleneimine and cellulose), which polymers have the advantage of being biocompatible, after the release of nitrogen oxide.

In one embodiment these polymers may be manufactured by electro spinning. Electro spinning is a process by which a polymer solution or melt is charged. At a characteristic voltage a fine jet of polymer releases from the surface in response to the tensile forces generated by interaction by an applied electric field with the electrical charge carried by the jet, on the way to the collector the jet stretches and solidifies, either by solvent evaporation or solidification. This process produces a non-woven mat of polymer fibers, such as nano-fibers. This jet of polymer fibers may be directed to a surface to be treated.

In one embodiment the polymers may be made into a solid powder, particles or granulate, before electro-spinning through a reaction with a strong base to a salt. The salt can be modified with NO. Said salt could be integrated in a polymer carrier, such as any suitable polymer, which polymers are mentioned below in respect of polymers suitable for mixing with nitric oxide eluting polymer, that could be electro spun according to the procedure described above. The advantage with this method is that the loading with NO of the said polymer could be done before electro-spinning.

Furthermore, U.S. Pat. No. 6,382,526, U.S. Pat. No. 6,520,425, and U.S. Pat. No. 6,695,992 disclose processes and apparatuses for the production of such polymeric fibers. These techniques are generally based on gas stream spinning, also known within the fiber forming industry as air spinning, of liquids and/or solutions capable of forming fibers.

Other example for NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be spun onto the surface of medical devices such as implanted grafts, showing significant improvement of the healing process and reduced inflammation when implanting such devices. For instance, U.S. Pat. No. 6,737,447 of Akron University discloses a coating for permanently implanted medical devices that provides nitric oxide delivery using nanofibers of linear poly(ethylenimine)-diazeniumdiolate. Linear poly(ethylenimine)-diazeniumdiolate releases nitric oxide (NO) in a controlled manner. Another advantage of L-PEI is that NO is released without any secondary products that could lead to undesired side effects. However, the meaning of controlled in the context of U.S. Pat. No. 6,737,447 is only directed to the fact that nitric oxide is eluted from the coating during a period of time, i.e. that the nitric oxide not is eluted all in once. Therefore, the interpretation of controlled in respect of U.S. Pat. No. 6,737,447 is different from the meaning of regulating in the present context. Regulate or control, according to the present context is intended to be interpreted as the possibility to vary the elution of nitric oxide to thereby achieve different elution profiles.

Three important factors in controlling and regulating the elution of nitric oxide from such a nitric oxide eluting polymer are how quickly a proton donor, such as body fluid in the urinary tract, comes in contact with the nitric oxide releasing polymer, such as a diazoliumdiolate group, the acidity of the environment surrounding the nitric oxide eluting polymer, and the temperature of the environment surrounding the nitric oxide releasing polymer (higher temperature promotes elution of nitric oxide).

A polymer comprising an O-nitrosylated group is also a possible nitric oxide eluting polymer. Thus, in an embodiment the nitric oxide eluting polymer comprises diazeniumdiolate groups, S-nitrosylated and O-nitrosylated groups, or any combinations thereof.

Some other examples of a suitable nitric oxide eluting polymer are selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

According to an embodiment, the nitric oxide (NO) eluting polymer comprises diazeniumdiolate groups, S-nitrosylated groups, and O-nitrosylated groups, or any combination of these.

In another embodiment the nitric oxide eluting polymer may be a NONOate. This kind of polymer does not need an enzymatic reaction to release nitric oxide.

In yet another embodiment said nitric oxide eluting polymer is a poly(alkyleneimine)-diazeniumdiolate, such as L-PEI-NO (linear poly(ethyleneimine)-diazeniumdiolate), where said nitric oxide eluting polymer is loaded with nitric oxide through the diazeniumdiolate groups and arranged to release nitric oxide at a treatment site. L-PEI offers the advantage of increased number of possible modification sites for NO loading.

Other ways of describing polymers, which may be suitable as embodiments of the herein discussed nitric oxide eluting polymer, is polymers comprising secondary amine groups (═N—H), such as L-PEI, or have a secondary amine (═N—H) as a pendant, such as aminocellulose.

The nitric oxide eluting polymer may comprise a secondary amine, either in the backbone or as a pendant, as described previously. This will make a good nitric oxide eluting polymer. The secondary amine should have a strong negative charge to be easy to load with nitric oxide. If there is a ligand close to the secondary amine, such as on a neighbor atom, such as a carbon atom, to the nitrogen atom, with higher electronegativity than nitrogen (N), it is very difficult to load the polymer with nitric oxide. On the other hand, if there is an electropositive ligand close to the secondary amine, such as on a neighbor atom, such as a carbon atom, to the nitrogen atom, the electronegativity of the amine will increase and thereby increase the possibility to load the nitric oxide elution polymer with nitric oxide.

In an embodiment the nitric oxide eluting polymer may be stabilized with a salt. Since the nitric oxide eluting group, such as a diazeniumdiolate group, usually is negative, a positive counter ion, such as a cation, may be used to stabilize the nitric oxide eluting group. This cation may for example be selected from the group comprising any cation from group 1 or group 2 in the periodic table, such as Na+, K+, Li+, Be2+, Ca2+, Mg2+, Ba2+, and/or Sr2+. Different salts of the same nitric oxide eluting polymer have different properties. In this way a suitable salt (or cation) may be selected for different purposes. Examples of cationic stabilized polymers are L-PEI-NO—Na, i.e. L-PEI diazeniumdiolate stabilized with sodium, and L-PEI-NO—Ca, i.e. L-PEI diazeniumdiolate stabilized with calcium.

With reference to the above general description of a proton donor being a part of regulating NO elution from a polymer, this fact is taken advantage of by some embodiments. Since the elution of nitric oxide is activated by a proton donor, such as water or body fluid, it may be an advantage to keep the nitric oxide eluting polymer, or eventually a mixture of nitric oxide eluting polymer and carrier material, in contact with said proton donor. If an indication requires an elution of nitric oxide during a prolonged period of time, a system is advantageous, which presents the possibility to keep the proton donor in contact with the nitric oxide eluting polymer, or mixture of nitric oxide eluting polymer and carrier material. A filling agent may give the nitric oxide eluting polymer, or mixture of said nitric oxide eluting polymer and a carrier material, a desired texture. Suitable filling agents may for instance be selected from the group comprising polyacrylates, polyethylene oxide, carboxymethylcellulose, and microcrystalline cellulose, cotton, and starch.

By providing an advantageous composition according to embodiments, a regulated, e.g. delayed, spontaneous, immediate or instantaneous, release of nitric oxide is provided in the urinary tract. For instance, the composition is composition configured to therapeutically target treat, prophylactically treat and/or prevent cancer and/or infection in a urinary tract, wherein said composition is configured to elute nitric oxide (NO) in a therapeutic dosage, and wherein said composition is configured to expose the urinary tract to said eluted nitric oxide. The composition comprises a nitric oxide (NO) eluting polymer configured for eluting a therapeutic dosage of nitric oxide (NO) configured for exposing an area of treatment in the urinary tract to said nitric oxide when said polymer in use elutes nitric oxide (NO). Furthermore, the nitric oxide (NO) eluting polymer may be integrated with a carrier material, such that said carrier material, in use, regulates and controls the elution of said therapeutic dosage of nitric oxide (NO) after elution is initiated.

In one embodiment a nitric oxide eluting polymer, such as L-PEI-NO, is mixed with a carrier polymer to slow down or prolong the elution of nitric oxide. Also, in another embodiment, the nitric oxide eluting polymer may be mixed with more than one carrier polymer, whereby be elution or release may be tailor made to fit specific needs. Such a need may for example be a low elution during a first period of time, when the environment of the nitric oxide eluting polymer is hydrophobic, and a faster elution during a second period of time, when the environment of the nitric oxide eluting polymer has been altered to be more hydrophilic. This may for example be accomplished by using biodegradable polymers, whereby a low elution during a first period of time is obtained, after which, when the hydrophobic polymer has been dissolved, the hydrophilic polymer provides a higher elution of nitric oxide. Thus, a more hydrophobic carrier polymer will give a slower elution of nitric oxide, since the activating proton donor, such as water or body fluid, will penetrate the carrier polymer slower. On the other hand, a hydrophilic polymer acts the opposite way. One example of an hydrophilic polymer is polyethylene oxide, and one example of an hydrophobic polymer is polystyrene. These carrier polymers may be mixed with the nitric oxide eluting polymer. The skilled person in the art knows which other polymers may be used for similar purposes.

In one embodiment this carrier polymer may have hydrophobic or hydrophilic properties. Therefore, the term carrier material in the present context should be interpreted to include carrier polymers and other materials with hydrophilic or hydrophobic properties.

In another embodiment the elution of nitric oxide from a nitric oxide eluting polymer, such as L-PEI-NO, is influenced by the presence of protons. This means that a more acidic environment provides a quicker elution of nitric oxide. By activating the nitric oxide eluting polymer, or mixture of nitric oxide eluting polymer and carrier material, with a pH-adjusting agent, e.g. an acidic fluid, such as an ascorbic acid solution, the elution of nitric oxide may be accelerated.

The carrier polymers and carrier materials mentioned above may affect other characteristics than the regulation of nitric oxide elution. An example of such characteristic is mechanical strength.

In respect of the carrier polymers or carrier materials, the NO-eluting polymer, such as in the form of powder, nano-particles, or micro-spheres, may be combined with, integrated in, spun together with, or spun on top of, any of these materials in some embodiments. In this way, one may manufacture a polymer mixture, comprising a nitric oxide eluting polymer and a carrier polymer, or a carrier material, with predefined nitric oxide eluting characteristics. These characteristics may be tailor made for different elution profiles in different applications.

The carrier polymer may be of any suitable polymer such as polyethylene, polyethylene oxide, polypropylene, polyacrylonitrile, polyurethane, polyvinylacetates, polylactic acids, starch, cellulose, carboxymethylcellulose, microcrystalline cellulose, polyhydroxyalkanoates, polyesters, polycaprolactone, polyvinylalcohol, polystyrene, polyethers, polycarbonates, polyamides, polyacrylates, poly(acrylic acid), Carboxy Methyl Cellulose (CMC), protein based polymers, gelatine, biodegradable polymers, cotton, starch, polyolefins, and latex, or any combinations of these.

In still another embodiment the nitric oxide eluting polymer, such as in form of a powder, nano-particles or micro-spheres, is incorporated in a foam. The foam may have an open cell structure, which facilitates the transport of the proton donor to the nitric oxide eluting polymer incorporated therein.

In an embodiment the composition is embodied in form of nano-particles, micro-spheres, or powder. These nano-particles, micro-spheres, or this powder may be formed from the NO-eluting polymers by spinning the NO eluting polymers into fibers, which fibers then are ground or milled into nano-particles, micro-spheres or powder. Alternative manufacturing methods or processes may be available. These nano-particles, micro-spheres, or this powder may the be injected into the urinary bladder, or applied in the urinary tract, in an amount sufficient to perform cancer treatment, prophylactic treatment, and treatment of infection. This injection may for example be done with a catheter through the urethra. A powder may also be applied directly to the urinary tract. When the nano-particles, micro-spheres, or powder get in contact with the moisture in the urinary bladder, the NO-eluting polymer starts to elute NO in the urinary bladder, to thereby subject the cancer cells and/or the infection to NO in such an amount as to achieve killing of said cancer cells and/or treating said infection.

In another embodiment the nano-particles, micro-spheres, or powder of NO eluting polymer is mixed before injection with a solvent, said solvent having a proton donor capability. The solvent with a proton donor capability may for example be selected from the group; water and/or alcohol, such as ethanol. In this embodiment the elution of NO starts when the nano-particles, micro-spheres, or powder of NO eluting polymer mixed with the solvent with proton donor capability. Therefore, it is preferred that the mixing of nano-particles, micro-spheres, or powder of NO eluting polymer and solvent is performed just prior to injection, to obtain as much elution of NO as possible inside the urinary bladder, or at other possible sites of therapy in the urinary tract. This injection may be performed with a catheter through the urethra.

In still another embodiment the nano-particles, micro-spheres, or powder of NO eluting polymer is mixed before injection with a hydrophobic solvent, for example hydrophobic cosmetic alcohol, such as lauryl alcohol. In this embodiment the elution of NO starts when the nano-particles, micro-spheres, or powder of NO eluting polymer gets in contact with the water or moisture in the urinary tract. This embodiment provides the advantage of being able to store the NO eluting composition in a slurry without initiating the elution of NO. It may also be possible to regulate and/or control the elution of NO to the area to be treated, for example the urinary tract of the patient suffering from cancer in the urinary tract. This embodiment may also be combined with the possibility to mix the hydrophobic solvent/nitric oxide eluting polymer mixture with a water based gel. Upon mixing this mixture will form an emulsion and start to elute nitric oxide. This emulsion also presents the possibility to regulate and/or control the elution of nitric oxide.

In another embodiment the nano-particles, micro-spheres, or powder may be formed from the NO-eluting polymers, encapsulated, or integrated, in any suitable material, such as polyvinylacetates, polylactic acids, starch, cellulose, polyhydroxyalkanoates, polycaprolactone, polyvinyl alcohol, protein based plastics, gelatine, biodegradable or biocompatible polymers, and other soluble plastics. The integration of, or encapsulation in, these materials is performed to regulate and/or control the elution of NO in the urinary tract, and to provide continuous exposure of the urinary tract to NO. The encapsulation may be such that the material breaks through reaction with the urine to thereby release the NO eluting polymer, which in contact with the water or moisture in the urinary tract starts to elute NO in the urinary tract of the patient suffering from cancer and/or infection, and/or prophylactic treatment in respect of said disorders, in said urinary tract.

In another embodiment the nano-particles, micro-spheres, or powder may be encapsulated in a suitable material, such as gelatine, starch, cellulose etc, to be introduced into the urinary tract, such as the urinary bladder, as a capsule. When the thus obtained capsule reaches the urinary bladder, the gelatine, starch, cellulose, etc., dissolves and the nano-particles, or micro-spheres, starts to elute NO to the urinary bladder.

In another embodiment the nano-particles, micro-spheres, or powder are/is compressed into a pill, tablet or pellet, which pill, tablet or pellet, then is introduced into the urinary tract, such as the urinary bladder, of the patient suffering from cancer in the urinary tract. When the pill, tablet, or pellet, has been introduced, an effective anti-cancer effect is initiated in said urinary tract, such as the urinary bladder, when the water or moisture in the urinary tract initiates elution of nitric oxide.

According to an embodiment, a composition is provided in a form selected from the group consisting of powder, nano-particles or micro-spheres, pill, tablet, pellet, gel, hydrogel, foam, cream, granules, capsule, solution, and suspension, or combinations thereof.

In yet another embodiment the NO-eluting composition may be combined with, or acting as a booster for, pharmaceuticals, chemotherapeutic agent, vitamins, nicotine, nitroglycerin etc. This embodiment presents a composition with the advantage of combining two therapeutic treatments, of significant value, in one treatment. A specific example of this embodiment is a combination of the composition according to certain embodiments and another active substance in respect of cancer, such as a chemotherapeutic substance. Hence, a synergetic effect may be achieved by such substances when NO that is eluted from the composition. NO has for instance a vasodilatory effect on the region where the composition acts. Thereby, the NO eluting composition in this embodiment may also facilitate the chemotherapeutic action of said chemotherapeutic substances, and hence achieving a synergistic effect. Vasodilated tissue is more susceptible to certain medications and thus more easily treated by the medical preparations and still NO has in addition to that the anti-cancer effect. Also, nitric oxide has an immunomodulating, cytotoxic and cytostatic effect. Nitric oxide acts as a signaling molecule in the human immune system. Thereby, nitric oxide can stimulate or inhibit different cells of the immune system depending on dose. This may in this embodiment be exploited to boost another chemotherapeutic substance or to counteract adverse effects of a chemotherapeutic substance. Furthermore, it happens that cancerous cells become resistant to a chemotherapeutic substance. In this context this embodiment combines the cytotoxic and/or cytostatic effect with the effect of said chemotherapeutic substance with a different mechanism of action to thereby achieve that the cancer cells will be less likely to develop resistance to said chemotherapeutic substance. Hence, an unexpected surprisingly effective treatment is provided.

Also, the effect of antibiotics in respect of treating infections, such as infections caused by bacteria and/or fungi, may be boosted and/or amplified by the use of nitric oxide in the same way as chemotherapeutic substances.

Urine is a unique environment in respect of treatment with nitric oxide, since the end oxidation product of nitric oxide is N02-. In almost all other biological tissues nitric oxide is oxidized to NO3-, since there is hemoproteins, such as hemoglobin, present. It has been shown that N02- may be reduced to NO in urine if pH is lowered and an antioxidant, such as ascorbic acid, is added.

Therefore, in one embodiment an antioxidant, such as ascorbic acid, is included in the NO eluting composition. A ten fold increase of this effect may be obtained if an antioxidant, such as ascorbic acid, is present.

The effect mentioned above, in respect of the unique environment of urine in treatment with NO, results in that very high concentrations of nitric oxide may be obtained during long periods of time in the urinary bladder, since the nitric oxide that is eluted in urinary bladder is first oxidized to N02-, which in return is reduced back to nitric oxide. This effect may be observed already at a pH of 7.5, but is strongly amplified at lower pH. A substantial amount of formed nitric oxide is obtained from N02- in urine at a pH of 4 to 5. All in all this results in the possibility to substantially potentiate the effects of a nitric oxide eluting system by simultaneously lowering the pH and adding an antioxidant.

Therefore, in another embodiment, pH is lowered in the urinary tract during treatment with the NO eluting composition. The lowering of the pH may for example be accomplished by addition of a pH-adjusting agent, such as ammonium chloride, ammonium sulphate, a biological acceptable acid, or a combination of several pH-adjusting agents.

In another embodiment of the composition, the nano-particles, micro-spheres, and/or powder, are integrated in a gel, such as a hydrogel. It may also be integrated in a hydrogel, which is mixed directly before use. This gel is then introduced into the urinary bladder by injection through the urethra, and the composition elutes NO when the gel, or hydrogel, reaches the urinary bladder. This gel may also be applied in for example the urethra or other sites of the urinary tract.

In yet another embodiment the composition is in form of a foam or cream.

When the NO-eluting composition according to certain embodiments gets in contact with the moisture or water in the urinary tract, the NO-eluting composition starts to release NO to the urinary tract to be treated. However, a delayed release is also feasible, e.g. by a coating dissolving by the influence of the moisture or water in the urinary tract, and the after a time delay giving access to the NO eluting composition. This composition does not cause resistance against nitric oxide (NO), is easy to apply, provides a painless treatment, has fast inset of treating and/or preventing anti-cancer effect and/or anti-infection effect.

In embodiments the NO eluting composition may be maintained in the urinary tract, such as the urinary bladder, of the patient suffering from cancer in said urinary tract until a sufficient time of treatment has been obtained. Then the composition may be discharged by urinating action of the patient or by discharging the composition through suction from the urinary bladder.

In yet another embodiment the NO eluting polymer is included in a catheter and/or catheter balloon to thereby provide the possibility to elute nitric oxide to the area of application of said catheter or catheter balloon. If such a catheter balloon is manufactured, e.g. of silicone, nitric oxide will be able to elute from the inside of the catheter balloon to the vicinity of the catheter balloon through the silicone wall of the catheter balloon.

In one embodiment ethambutol is used as the carrier of nitric oxide, to thereby constitute a nitric oxide eluting substance. In this embodiment nitric oxide is eluted in the same way as with the nitric oxide eluting polymers discussed above.

The treatment with the composition according to embodiments is very effective in respect of treating both cancer in the epithelial cells (the internal lining), such as superficial cancer in the urinary bladder, and cancer in the muscle layer of the urinary bladder. This combinatory effect is achieved through the anti-cancer effect of NO and the vasodilating effect of NO.

The device comprising the composition according to embodiments, or the composition elutes in use nitric oxide (NO) from said eluting polymer in a therapeutic dose in the urinary tract, such as between 0.001 to 5000 ppm, such as 0.01 to 3000 ppm, such as 0.1 to 1000 ppm, such as 1 to 100 ppm, for instance 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the actual NO eluting polymer the concentration may be as high as thousands of ppm, while the concentration inside the tissue in this case often is considerably lower, such as between 0.1 to 100 ppm.

The NO-eluting polymers in the composition according to embodiments may be combined with silver, such as hydro-activated silver. The integration of silver in the composition according to this present embodiment gives the healing process an extra boost. Preferably the silver is releasable from the composition in the form of silver ions.

In certain embodiments it may be provided to control or regulate the time span of NO release from the composition. This may be accomplished by integrating other polymers or materials in said composition. These polymers or materials may be chosen from any suitable material or polymer, such as polyvinylacetates, polylacticacids, starch, cellulose, polyhydroxyalkanoates, polyesters, polycaprolactone, polyvinylalcohol, protein based plastics, gelatine, biodegradable polymers, and other soluble plastics.

The composition according to embodiments may comprise polymers manufactured by, for example electro spinning of L-PEI or other polymers comprising L-PEI or being arranged in combination with L-PEI. L-PEI is the charged at a characteristic voltage, and a fine jet of L-PEI releases as a bundle of L-PEI polymer fibers. The L-PEI polymer fibers may be spun onto any suitable material in respect of certain embodiments. The electro spun fibers of L-PEI then attach on said material from which the nano-particles, micro-spheres, or powder are/is formed. Such fibers are also easily further processed to other forms, such as powder, which simply is obtainable by applying a shredder or blender type apparatus to the fibers until a powder of desired granulation size is received.

However, powder may also be obtained by other methods. For instance, polymers may be made into a solid powder, particles or granulate, through a reaction with a strong base to a salt during modification with NO. Said salt could be integrated in a polymer carrier, such as any suitable polymer, which polymers are mentioned below in respect of polymers suitable for mixing with nitric oxide eluting polymer, that could be electro spun or otherwise manufactured. The advantage with this method is that the loading with NO of the said polymer could be done before manufacturing.

It is of course possible to electro spin the other NO-eluting polymers, according to above, on the thus obtained NO eluting polymers, which may be comprised in the compositions according to embodiments of the present invention, while still be inside the scope of the present invention.

In one embodiment the NO-eluting polymers are electro spun in such way that pure NO-eluting polymer fibers may be obtained.

Gas stream spinning, air spinning, wet spinning, dry spinning, melt spinning, or gel spinning, of said NO-eluting polymers onto material, which combination of NO-eluting polymer and other material may be comprised in the composition according to certain embodiments, is also within the scope of an embodiment of the manufacturing method.

According to an embodiment, the manufacturing process further comprises selecting a nitric oxide (NO) eluting polymer configured to elute a therapeutic dosage of nitric oxide (NO) when used for said therapeutically target treatment, prophylactically treatment and/or prevention of cancer and/or infection in the urinary tract, selecting a carrier material, which carrier material is configured to regulate and control the elution of said therapeutic dosage of nitric oxide (NO), incorporating the NO-eluting polymer with said carrier material into an nitric oxide (NO) eluting material, such that said carrier material, in use of said device, regulates and controls the elution of said therapeutic dosage of nitric oxide (NO), and deploying said nitric oxide eluting material into a suitable form, or as a coating onto a carrier, to form at least a part of said device, such that said device is configured to expose a therapeutic target site to said nitric oxide when said NO-eluting polymer in use elutes nitric oxide (NO).

According to another embodiment, the manufacturing process further comprises selecting a nitric oxide (NO) eluting polymer configured to elute a therapeutic dosage of nitric oxide (NO) when used for said therapeutically target treatment, prophylactically treatment and/or prevention of cancer and/or infection in the urinary tract, selecting a liquid material, which liquid material is configured to regulate and control the elution of said therapeutic dosage of nitric oxide (NO), incorporating the NO-eluting polymer with said liquid material into an nitric oxide (NO) eluting material, such that said liquid material, in use of said device, regulates and controls the elution of said therapeutic dosage of nitric oxide (NO), and deploying said nitric oxide eluting material into a solution or suspension with said liquid material, to form at least a part of said device, such that said device is configured to expose a therapeutic target site to said nitric oxide when said NO-eluting polymer in use elutes nitric oxide (NO).

In yet a further embodiment, the manufacturing process comprises selecting a plurality of nitric oxide (NO) eluting polymeric particles, such as nano-fibers, nano-particles, micro-spheres or powder, for said nitric oxide eluting polymer.

In another embodiment, the manufacturing comprises integrating said NO-eluting polymer in a carrier material, spinning said NO-eluting polymer together with said carrier material, or spinning said NO-eluting polymer on top of said carrier material, in order to predefine nitric oxide eluting characteristics of said composition.

Also, the manufacturing process may comprise integrating silver in said device, or selecting a pharmaceutical, vitamin, drug or a combination of theses, for which said nitric oxide (NO) eluting polymer is configured to act as a booster.

The manufacturing process presents the advantages of providing compositions with large contact surface of the NO-eluting polymer fibers and with the area to be treated, effective use of NO-eluting polymer, and a cost effective way of producing the composition according to certain embodiments of the present invention.

Hereinafter, some potential uses of some embodiments of the present invention are described:

A method of therapeutical treatment of cancer in the urinary tract by means of a composition comprising a nitric oxide (NO) eluting polymer configured for eluting a therapeutic dosage of nitric oxide (NO) when used for said treatment, comprising exposing said treatment site of said cancer in the urinary tract to said nitric oxide when said polymer in use elutes nitric oxide (NO) by eluting a therapeutic dose of nitric oxide from said nitric oxide eluting polymer to said urinary tract.

The method may further comprise lowering the pH in the urinary tract, or adding an antioxidant to said urinary tract.

The method according to the above, wherein said method comprises applying nano-particles, micro-spheres, or powder as a pill, a tablet, a pellet, a capsule, composition, a gel, a hydrogel, a foam, a cream, and/or granules, to said site for said exposure.

Use of nitric oxide (NO) in a therapeutic dose for therapeutically treating cancer in the urinary tract.

Use of a nitric oxide (NO) eluting polymer for the manufacture of a composition for the treatment and/or prevention of cancer and/or infection in the urinary tract wherein nitric oxide is loaded to said composition so that said composition elutes nitric oxide (NO) from said eluting polymer in a therapeutic dose when used.

Use of a nitric oxide (NO) eluting polymer, wherein said composition is a composition according to certain embodiments described herein above.

Nitric oxide (NO) may be used as a medicament in the treatment of cancer in the urinary tract.

Nitric oxide (NO) may be used as a medicament in the treatment of cancer in the epithelial cells in the urinary tract.

Nitric oxide (NO) may be used as a medicament in the treatment of infection in the urinary tract.

Nitric oxide (NO) may be used in a therapeutic dose for therapeutically treating cancer in the urinary tract.

A composition according to embodiments described above may be used for the treatment and/or prevention of cancer and/or infection in the urinary tract.

The invention may be implemented in any suitable form. The elements and components of the embodiments according to the invention may be physically, functionally, and logically implemented in any suitable way. Indeed, the functionality may be implemented in a single unit, in a plurality of units, or as part of other functional units.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc. do not preclude a plurality. Reference signs in the claims are provided merely as a clarifying example and shall not be construed as limiting the scope of the claims in any way.

The present invention has been described above with reference to specific embodiments. However, other embodiments than the above described are equally possible within the scope of the invention. Different method steps than those described above, performing the method by hardware or software, may be provided within the scope of the invention. The different features and steps of the invention may be combined in other combinations than those described. The scope of the invention is only limited by the appended patent claims.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. Nitric oxide (NO) for use as a medicament in the treatment of cancer in the urinary tract.

2. Nitric oxide (NO) for use as a medicament in the treatment of cancer in the epithelial cells in the urinary tract.

3. Nitric oxide (NO) for use as a medicament in the treatment of infection in the urinary tract.

4. Use of a nitric oxide (NO) eluting polymer for the manufacture of a composition for the treatment and/or prevention of cancer and/or infection in the urinary tract wherein nitric oxide is loaded to said composition so that said composition elutes nitric oxide (NO) from said eluting polymer in a therapeutic dose when used.

5. Use according to claim 4, wherein said composition is a composition configured to therapeutically target treat, prophylactically treat and/or prevent cancer and/or infection in a urinary tract, wherein
said composition is configured to elute nitric oxide (NO) in a therapeutic dosage, and wherein said composition is configured to expose the urinary tract to said eluted nitric oxide,
and wherein said composition comprises a nitric oxide (NO) eluting polymer configured to elute a therapeutic dosage of nitric oxide (NO) to expose an area of treatment in the urinary tract to said nitric oxide when said polymer in use elutes nitric oxide (NO), and
wherein elution of said therapeutic dosage of nitric oxide (NO) from said nitric oxide (NO) eluting polymer is regulateable.

6. Use according to claim 4 or 5, wherein said therapeutic dose is in the range of 0.001 to 5000 ppm, such as 0.01 to 3000 ppm, such as 0.1 to 1000 ppm, such as 1 to 100 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm.

7. A medical device comprising a composition, wherein the medical device is a urinary catheter comprising a balloon, characterized by
said balloon having a permeable wall, wherein said composition is
a composition configured to therapeutically target treat, prophylactically treat and/or prevent cancer and/or infection in a urinary tract, wherein
said composition is configured to elute nitric oxide (NO) in a therapeutic dosage, and wherein said composition is configured to expose the urinary tract to said eluted nitric oxide, and wherein
said composition comprises a nitric oxide (NO) eluting polymer configured to elute a therapeutic dosage of nitric oxide (NO) to expose an area of treatment in the urinary tract to said nitric oxide when said polymer in use elutes nitric oxide (NO), and
wherein elution of said therapeutic dosage of nitric oxide (NO) from said nitric oxide (NO) eluting polymer is regulateable, and wherein
said wall is permeable for said nitric oxide (NO) such that said nitric oxide (NO) upon application of said urinary catheter is eluted, from inside of the catheter balloon and from said composition to said urinary tract through said wall.

8. Medical device according to claim 7, wherein said wall is of silicone.

9. Medical device according to claim 7 or 8, wherein said regulateable elution of nitric oxide (NO) comprises prolonged, delayed, spontaneous, instantaneous or immediate, elution of nitric oxide from said nitric oxide (NO) eluting polymer.

10. Medical device according to any of claims 7 to 9, wherein said elution of said therapeutic dosage of nitric oxide (NO) from said nitric oxide (NO) eluting polymer is regulateable by means of a carrier material.

11. Medical device according to claim 10, wherein said nitric oxide (NO) eluting polymer is integrated with a carrier material, such that said carrier material, in use, regulates and controls the elution of said therapeutic dosage of nitric oxide (NO).

12. Medical device according to any of claims 7 to 9, wherein said nitric oxide (NO) eluting polymer comprises diazeniumdiolate groups, S-nitrosylated groups, and O-nitrosylated groups, or any combination of these.

13. Medical device according to any of claims 7 to 9, or 12, wherein said nitric oxide (NO) eluting polymer is L-PEI (linear polyethyleneimine), loaded with nitric oxide (NO) through said diazeniumdiolate groups, S-nitrosylated groups, or O-nitrosylated groups, or any combination these, arranged for release of said nitric oxide (NO) to an adjacent mammal tissue in said urinary tract.

14. Medical device according to any of claims 7 to 9, wherein said nitric oxide eluting polymer is selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

15. Medical device according to any of claims 7 to 9, wherein said nitric oxide eluting polymer comprises a secondary amine in the backbone or a secondary amine as a pendant.

16. Medical device according to claim 15, wherein a positive ligand is located on the neighbor carbon atom to the secondary amine.

17. Medical device according to any of claims 7 to 9, wherein said elution of said therapeutic dosage of nitric oxide (NO) from said nitric oxide (NO) eluting polymer is regulateable by means of a cation.

18. Medical device according to any of claims 7 to 9 or claim 17, wherein said composition comprises a cation, said cation stabilizing the nitric oxide eluting polymer.

19. Medical device according to claim 18, wherein said cation is selected from the group comprising Na+, K+, Li+, Be2+, Ca2+, Mg2+, Ba2+, and/or Sr2+, or any combinations thereof.

20. Medical device according to any of claims 7 to 19, wherein said polymer is L-PEI (linear polyethyleneimine), loaded with nitric oxide (NO), arranged for release of the nitric oxide (NO) at said urinary tract.

21. Medical device according to any of claims 7 to 20, wherein said composition comprises an antioxidant.

22. Medical device according to claim 21, wherein said antioxidant is ascorbic acid.

23. Medical device according to any of claims 7 to 22, wherein said composition comprises a pH lowering substance.

24. Medical device according to claim 23, wherein said pH lowering substance is chosen from the group consisting of ammonium chloride, ammonium sulphate, a biologically acceptable acid, or a combination of these.

25. Medical device according to any of claims 7 to 24, further comprising a pH-adjusting agent, for lowering the pH value in the urinary tract.

26. Medical device according to claim 25, wherein said pH-adjusting agent is chosen from the group consisting of ammonium chloride, ammonium sulphate, a biological acceptable acid, or a combination of several pH-adjusting agents.

27. Medical device according to any of claims 7 to 26, wherein said composition comprises nitric oxide (NO) eluting ethambutol.

28. Medical device according to any of claims 7 to 27, wherein said composition is in a form selected from the group consisting of powder, nano-particles or micro-spheres, pill, tablet, pellet, gel, hydrogel, foam, cream, granules, capsule, solution, and suspension, or combinations thereof.

29. Medical device according to claim 28, wherein said nano-particles, micro-spheres, granules, or powder, are encapsulated, combined with, or integrated, in a material, selected from the group consisting of polyvinylacetates, polylacticacids, starch, cellulose, polyhydroxyalkanoates, polyesters, polycaprolactone, polyvinylalcohol, protein based plastics, and/or gelatine, polyesters, polyamides, polyethers, polyurethanes, polycarbonates, polyvinylacetates, polylacticacids, starch, cellulose, polyhydroxyalkanoates, polypropylene, cotton, polyesters, polycaprolactone, polyvinylalcohol, polyacrylonitrile, polystyrene, poly(acrylic acid), polypropylene, protein based plastics, gelatine, and other biocompatible polymers, and combinations thereof, for regulating and/or controlling elution of NO.

30. Medical device according claim 28, wherein said nano-particles, micro-spheres, are or said powder is integrated in a gel, hydrogel, pill, tablet, capsule, foam, solution, or suspension, and combinations thereof.

31. Medical device according claim 28, wherein said granules, nano-particles, micro-spheres, are or said powder is of a material, selected from the group consisting of polyesters, polyamides, polyethers, polyurethanes, polycarbonates, polyvinylacetates, polylacticacids, starch, cellulose, polyhydroxyalkanoates, polypropylene, cotton, polyesters, polycaprolactone, polyvinylalcohol, polyacrylonitrile, polystyrene, poly(acrylic acid), polypropylene, protein based plastics, gelatine, and other biocompatible polymers, and combinations thereof, integrated, or covered, with said NO-eluting polymer.

32. Medical device according to any of claims 7 to 9 or 28, wherein said elution of said therapeutic dosage of nitric oxide (NO) from said nitric oxide (NO) eluting polymer is regulateable by means of a physical form or shape of said nitric oxide (NO) eluting polymer.

33. Medical device according to any of claims 7 to 32, wherein said composition is configured to elute NO when subjected to a proton donor.

34. Medical device according to claim 33, wherein said proton donor is moisture, water, or a body fluid in said urinary tract.

35. Medical device according to claim 33, wherein said composition is configured to immediately elute said nitric oxide upon contact with said proton donor in said urinary tract.

36. Medical device according to any of claims 7 to 9 or 33, wherein said elution of said therapeutic dosage of nitric oxide (NO) from said nitric oxide (NO) eluting polymer is regulateable by means of a proton donor.

37. Medical device according to any of the preceding claims 7 to 36, wherein said nitric oxide (NO) eluting polymer further is combined with silver, configured for said therapeutical target treatment, prophylactical treatment and/or prevention of cancer and/or infection in the urinary tract.

38. Medical device according to any of the preceding claims 7 to 37, wherein said nitric oxide (NO) eluting polymer is configured to act as a booster for pharmaceuticals, vitamins, and drugs, or combinations thereof.

39. Medical device according to claims 7 to 9 or claim 38, wherein said composition is combined with silver.

40. Medical device according to any of the preceding claims 7 to 39, wherein said composition is combined with a chemotherapeutic agent.

41. Medical device according to any of the preceding claims 7 to 40, wherein said composition comprises a hydrophobic alcohol.

42. Medical device according to claim 41, wherein said hydrophobic alcohol is lauryl alcohol.

43. A manufacturing process for a composition comprised in said medical device according to any of the preceding claims 7 to 42, said manufacturing process comprising
selecting a plurality of nitric oxide eluting polymeric particles, such as nano-fibers, nano-particles or micro-spheres, and
deploying said nitric oxide eluting polymeric particles into a suitable form or as a coating onto a carrier, to form a material comprised in said composition.

44. Manufacturing process according to claim 43, further comprising
selecting a nitric oxide (NO) eluting polymer configured to elute a therapeutic dosage of nitric oxide (NO) when used for said therapeutically target treatment, prophylactically treatment and/or prevention of cancer and/or infection in the urinary tract,
selecting a carrier material, which carrier material is configured to regulate and control the elution of said therapeutic dosage of nitric oxide (NO),
incorporating the NO-eluting polymer with said carrier material into an nitric oxide (NO) eluting material, such that said carrier material, in use of said device, regulates and controls the elution of said therapeutic dosage of nitric oxide (NO), and
deploying said nitric oxide eluting material into a suitable form, or as a coating onto a carrier, to form at least a part of said device, such that said device is configured to expose a therapeutic target site to said nitric oxide when said NO-eluting polymer in use elutes nitric oxide (NO).

45. The manufacturing process according to claim 43 or 44,
wherein said deploying comprises electro spinning, air spinning, gas spinning, wet spinning, dry spinning, melt spinning, or gel spinning of NO-eluting polymer.

46. Manufacturing process according to claim 43, further comprising
selecting a nitric oxide (NO) eluting polymer configured to elute a therapeutic dosage of nitric oxide (NO) when used for said therapeutically target treatment, prophylactically treatment and/or prevention of cancer and/or infection in the urinary tract,
selecting a liquid material, which liquid material is configured to regulate and control the elution of said therapeutic dosage of nitric oxide (NO),
incorporating the NO-eluting polymer with said liquid material into an nitric oxide
(NO) eluting material, such that said liquid material, in use of said device, regulates and controls the elution of said therapeutic dosage of nitric oxide (NO), and
deploying said nitric oxide eluting material into a solution or suspension with said liquid material, to form at least a part of said device, such that said device is configured to expose a therapeutic target site to said nitric oxide when said NO-eluting polymer in use elutes nitric oxide (NO).

47. The manufacturing process according to any of claims 43 to 46, wherein said selecting said nitric oxide (NO) eluting polymer comprises selecting a plurality of nitric oxide (NO) eluting polymeric particles, such as nano-fibers, nano-particles, micro-spheres or powder.

48. The manufacturing process according to any of claims 43 to 47, wherein said incorporating said NO-eluting polymer with said carrier material comprises integrating said NO-eluting polymer in said carrier material, spinning said NO-eluting polymer together with said carrier material, or spinning said NO-eluting polymer on top of said carrier material, in order to predefine nitric oxide eluting characteristics of said device.

49. The manufacturing process according to any of claims 43 to 48, further comprising integrating silver in said device.

50. The manufacturing process according to any of claims 43 to 49, further comprising selecting a pharmaceutical, vitamin, drug or a combination of theses, for which said nitric oxide (NO) eluting polymer is configured to act as a booster.

51. A method of a treatment and/or prevention of cancer and/or infection in the urinary tract, comprising using a composition comprising a nitric oxide (NO) eluting polymer configured for eluting a therapeutic dosage of nitric oxide (NO) when used for said treatment and/or prevention, comprising exposing a site in the urinary tract to said nitric oxide when said polymer in use elutes nitric oxide (NO) by eluting a therapeutic dose of nitric oxide from said nitric oxide eluting polymer to said urinary tract.

52. The method according to claim 51, wherein said method further comprises lowering the pH in the urinary tract during treatment with the NO eluting composition.

53. The method according to claim 52, comprising
accomplishing said lowering of the pH value by addition of a pH-adjusting agent, such as ammonium chloride, ammonium sulphate, a biological acceptable acid, or a combination of several pH-adjusting agents.

54. The method according to claim 51 comprising injecting said composition a catheter through the urethra to the urinary tract, such as to the bladder.

55. The method according to claim 54, wherein said composition is in a form selected from the group consisting of powder, nano-particles or micro-spheres, said method comprising
mixing said nano-particles or micro-spheres before said injecting with a solvent, said solvent having a proton donor capability, and
obtaining elution of NO inside the urinary tract.

56. The method according to claim 54, wherein said composition is in a form selected from the group consisting of powder, nano-particles or micro-spheres, said method comprising
mixing said nano-particles or micro-spheres before said injecting with a hydrophobic solvent, and
starting the elution of NO starts when the nano-particles, micro-spheres, or powder of NO eluting polymer gets in contact with the water or moisture in the urinary tract.

57. The method according to claim 56, comprising mixing said hydrophobic solvent/nitric oxide eluting polymer mixture with a water based gel for forming an emulsion to regulate and/or control the elution of nitric oxide.

58. The method according to claim 51, wherein said composition is in a form selected from the group consisting of powder, nano-particles or micro-spheres, encapsulated, or integrated, in a material, and
dissolving or breaking said encapsulation material in the urinary tract to start eluting said NO in the urinary tract of the patient for said treatment and/or prevention of cancer and/or infection in the urinary tract.

59. The method according to claim 51, wherein said method comprises applying nano-particles, micro-spheres, or powder as a pill, a tablet, a pellet, a capsule, composition, a gel, a hydrogel, a foam, a cream, and/or granules, to said site for said exposure.

60. The method according to any of claims 51 to 59, wherein said method further comprises adding an antioxidant to said urinary tract.

61. The method according to claim 51, comprising said NO-eluting composition acting as a booster for, pharmaceuticals, chemotherapeutic agent, vitamins, nicotine, or nitroglycerin.

62. The method according to claim 51, comprising
stimulating or inhibiting different cells of the immune system for boosting a chemotherapeutic substance or to counteract adverse effects of the chemotherapeutic substance.

63. The method according to claim 51, wherein said eluting of said nitric oxide (NO) comprises,
applying a urinary catheter in said urinary tract, said urinary catheter having a catheter balloon comprising a wall that is permeable for said nitric oxide (NO), and
eluting said nitric oxide (NO) from said composition to said urinary tract through said wall.

64. Use of nitric oxide (NO) in a therapeutic dose for therapeutically treating cancer in the urinary tract.

65. Use of a composition for a treatment and/or prevention of cancer and/or infection in a urinary tract, wherein said composition is configured to therapeutically target treat, prophylactically treat and/or prevent cancer and/or infection in the urinary tract, wherein
said composition is configured to elute nitric oxide (NO) in a therapeutic dosage, and wherein said composition is configured to expose the urinary tract to said eluted nitric oxide, and wherein
said composition comprises a nitric oxide (NO) eluting polymer configured to elute a therapeutic dosage of nitric oxide (NO) to expose an area of treatment in the urinary tract to said nitric oxide when said polymer in use elutes nitric oxide (NO), and
wherein elution of said therapeutic dosage of nitric oxide (NO) from said nitric oxide (NO) eluting polymer is regulateable.
